(19) United States
(12) Patent Application Publication
(10) Pub. No.: US 2019/0183379 A1
(43) Pub. Date: Jun. 20, 2019

(54) MEDICAL DEVICE LOCATION AND TRACKING SYSTEM

(71) Applicant: Medical Components, Inc., Harleysville, PA (US)

(72) Inventor: Andres Bagalawis Cruz, JR., Red Hill, PA (US)

(21) Appl. No.: 16/226,866

(22) Filed: Dec. 20, 2018

Related U.S. Application Data
(60) Provisional application No. 62/608,160, filed on Dec. 20, 2017.
(60) Provisional application No. 62/725,527, filed on Aug. 31, 2018.

(51) Int. Cl. A61B 5/06; A61B 5/042; A61B 5/044; A61B 5/0452

(52) U.S. Cl. CPC A61B 5/062; A61B 5/042; A61M 2025/0166; A61B 5/0452; A61B 5/063; A61B 5/044

(57) ABSTRACT

Embodiments can relate to a system configured for locating and tracking the movement of a medical device (e.g., a catheter). Embodiments of the system can include use of energized induction coil assemblies located outside of a patient to generate an electronic field and/or a magnetic field to induce signals in an inductive sensor located in the tip of the catheter and/or stylet. The sensor signals can be analyzed to provide data related to the position and trajectory of the catheter tip and/or stylet as it is moved within the body. In some embodiments, the energized induction coil assemblies can be disposed in and/or on a platform that is placed on a patient's chest.

### CROSS-REFERENCE TO RELATED APPLICATIONS

This application claims priority to U.S. Provisional Application Nos. 62/608,160, filed Dec. 20, 2017, and 62/725,527, filed Aug. 31, 2018, the contents of which are incorporated by reference herein.

### FIELD OF THE INVENTION

Systems and methods disclosed herein relate to detection and tracking of a medical device (e.g., catheter) inserted into a patient that can provide an orientation, location, and/or movement data for placement, positioning, and guidance of the medical device.

### BACKGROUND OF THE INVENTION

Conventional medical device location and tracking systems can be appreciated from U.S. Pat. Nos. 4,173,228, 4,317,078, 4,445,501, 4,905,698, 5,005,592, 5,099,845, 5,211,165, 5,381,795, 5,386,828, 5,412,619, 5,640,960, 5,645,065, 8,380,289, 8,388,541, and U.S. 2010/0222664. Conventional systems may be inaccurate and inefficient. Additionally, conventional systems may be limited in functionality and versatility. These and other disadvantages may limit the use of conventional location and tracking systems.

### BRIEF SUMMARY OF THE INVENTION

Embodiments can relate to a system configured for locating and tracking the movement of a medical device (e.g., a catheter). Embodiments of the system can include use of energized induction coil assemblies located outside of a patient to generate an electronic field and/or a magnetic field to induce signals in an inductive sensor located in the tip of the catheter. The sensor signals can be analyzed to provide data related to the position and trajectory of the catheter tip as it is moved within the body. In some embodiments, the energized induction coil assemblies can be disposed in and/or on a platform that is placed on a patient's chest.

Some embodiments can include use of electrocardiograph (“ECG”) signals to assist with location and tracking of the catheter tip by analyzing a waveform exhibited in the ECG signal. Inflection points of waveforms (e.g., P-waves) can be used to determine the relative distance between the catheter tip and the heart.

Some embodiments can include use of an ultrasound imaging unit that emits ultrasound signals via a handheld probe to facilitate generating an ultrasound image of the catheter tip. The ultrasound probe can be configured to emit ultrasound signals directed toward the catheter tip located in the body of a patient. The reflected ultrasound signals can be detected by transducer arrays and analyzed.

The data from any one or a combination of the location and tracking techniques disclosed herein can be displayed via a computer device as an image, graphical data, tabular data, etc.

In at least one embodiment, a medical device location and tracking system can include a plurality of induction coil assemblies configured to be placed outside of a patient's body. Each induction coil assembly may be configured to generate an electric field and/or a magnetic field (EM field) that permeates at least a portion of the patient's body. At least one EM field may be perpendicular or orthogonal to another EM field. The system may include an inductive sensor configured to be inserted into a patient. The inductive sensor can be configured to generate a sensor signal based on at least one of: a magnitude of the EM field; a direction of the EM field; and a flux of the EM field. The system can include an electrical power source configured to energize each induction coil assembly of the plurality of induction coil assemblies in a sequential manner.

In some embodiments, at least one induction coil assembly can be configured to generate an EM field having a magnitude that is different from a magnitude of an EM field generated by another induction coil assembly. In some embodiments, the plurality of induction coil assemblies can include a first induction coil assembly, a second induction coil assembly, a third induction coil assembly, and a fourth induction coil assembly. In some embodiments, the EM field of each of the first induction coil assembly, the second induction coil assembly, and the third induction coil assembly can be facing a first direction, and the EM field of the fourth induction coil assembly is facing a second direction. In some embodiments, the first direction is perpendicular or orthogonal to the second direction. In some embodiments, the EM field of each of the first induction coil assembly, the second induction coil assembly, and the third induction coil assembly can be set at a first magnitude, and the EM field of the fourth induction coil assembly is set at a second magnitude. In some embodiments, the first magnitude can be greater than the second magnitude.

In some embodiments, the plurality of induction coil assemblies can include a first induction coil assembly, a second induction coil assembly, a third induction coil assembly, a fourth induction coil assembly, and a fifth induction coil assembly. In some embodiments, the EM field of each of the first induction coil assembly, the second induction coil assembly, and the third induction coil assembly can be facing a first direction, the EM field of the fourth induction coil assembly can be facing a second direction, and the EM field of the fifth induction coil assembly can be facing a third direction. In some embodiments, the first direction can be perpendicular or orthogonal to the second direction and can be perpendicular or orthogonal to the third direction. In some embodiments, the EM field of each of the first induction coil assembly, the second induction coil assembly, and the third induction coil assembly can be set at a first magnitude, and each of the EM fields of the fourth induction coil assembly and fifth induction coil assembly can be set at a second magnitude. In some embodiments, the first magnitude can be greater than the second magnitude.

In some embodiments, the induction coil assembly can include at least one sub-induction coil. In some embodiments, the induction coil assembly can include a plurality of sub-induction coils. In some embodiments, the plurality of sub-induction coils can include a first sub-induction coil facing a first direction and a second sub-induction coil facing a second direction. In some embodiments, the first sub-induction coil direction can be perpendicular or orthogonal to the second sub-induction coil direction.

In some embodiments, the plurality of sub-induction coils can include a first sub-induction coil facing a first direction, a second sub-induction coil facing a second direction, and a third sub-induction coil facing a third direction. In some embodiments, the first sub-induction coil direction can be perpendicular or orthogonal to the second sub-induction coil direction and can be perpendicular or orthogonal to the third sub-induction coil direction.

In some embodiments, the system can further include a pair of electrocardiograph (“ECG”) electrodes placed on the patient's body. The system can further include an ECG sensor electrode placed in and/or on the medical device. The pair of ECG electrodes and the ECG sensor electrode can be configured to produce ECG signals that are transmitted to an electrocardiogram display. Changes in a P-wave of the ECG signals can indicate movement of the medical device towards or away from a heart of a patient.

In at least one embodiment, a medical device location and tracking system can include a platform configured to be positioned outside a patient's body. The platform can include a first induction coil assembly, a second induction coil assembly, and a third induction coil assembly. The platform can also include a plurality of inter-disposed induction coil assemblies. Each of the first induction coil assembly, the second induction coil assembly, the third induction coil assembly, and the inter-disposed induction coil assembly can be configured to generate an electric field and/or a magnetic field (EM field) that permeates at least a portion of the patient's body. At least one EM field of the inter-disposed induction coil assembly can be perpendicular or orthogonal to at least one EM field of the first induction coil assembly, the second induction coil assembly, the third induction coil assembly. The system can further include a power source configured to energize at least two of the first induction coil assembly, the second induction coil assembly, the third induction coil assembly, and the inter-disposed induction coil assembly in a sequential manner. The system can further include an inductive sensor disposed in and/or on a portion of a medical device configured to be inserted into the patient. The inductive sensor may be configured to generate a sensor signal based on being within and/or proximal to any one or a combination of the EM fields generated by the first induction coil assembly, the second induction coil assembly, the third induction coil assembly, and the inter-disposed induction coil assembly.

In at least one embodiment, a method for locating and tracking a medical device can involve positioning a plurality of induction coil assemblies on top of and/or over a patient's body. The method can involve generating a plurality of electric fields and/or magnetic fields (EM fields) that permeate at least a portion of the patient's body by energizing at least two of the induction coil assemblies in a sequential manner. The method can involve inserting the medical device with an inductive sensor into the patient. The inductive sensor may be configured to generate a sensor signal based on at least one of: a magnitude of at least one EM field; a direction of at least one EM field; and a flux of at least one EM field. The method can involve recording a plurality of sensor signals. The method can involve determining a location of the medical device based on a proximity with which the inductive sensor is relative to at least one of the induction coil assemblies.

In at least one embodiment, a method for locating and tracking a medical device can involve positioning a plurality of induction coil assemblies on top of and/or over a patient's body. The method can involve generating a plurality of electric fields and/or magnetic fields (EM fields) that permeate at least a portion of the patient's body by energizing at least one of the induction coil assemblies. The method can involve inserting the medical device with an inductive sensor into the patient. The method can involve determining a location of the medical device based on a proximity with which the inductive sensor is relative to at least one of induction coil assemblies. The method can involve tracking movement of the medical device to determine if the medical device is taking a superior vena cava path 1, a jugular path 2, or a path that is across a left brachiocephalic 3 of the patient. (See FIGS. 10-11)

In at least one embodiment, a platform for a location and tracking system can include a structure configured to be placed on top of or over a patient's body. In some embodiments, the platform can include a plurality of induction coil assemblies disposed in and/or on a portion of the structure. In some embodiments, the structure can be configured to hold each induction coil assembly at a predetermined position and/or orientation. In some embodiments, the orientation of at least one induction coil assembly can be orthogonal or perpendicular to at least one other induction coil assembly.

In some embodiments, the structure can include a T-shape. In some embodiments, the structure the T-shaped structure can include an arm having a first arm end and a second arm end. The structure may also include a stem having a first stem end and a second stem end. The plurality of induction coil assemblies may include a first induction coil assembly disposed on the arm at or near the first arm end, a second induction coil assembly disposed on the arm at or near the second arm end, and a third induction coil assembly disposed on the stem, at or near the second stem end. Some embodiments of the platform can include at least one of an inter-disposed induction coil assembly disposed: a) on the arm between the first arm end and the second arm end; and b) on the stem between the first stem end and the second stem end.

In at least one embodiment, a method for tracking a medical device can include inserting the medical device into a patient. The method can further include determining a location of the medical device. The method can further include tracking movement of the medical device to determine if the medical device is taking a superior vena cava path, a jugular path, or a path that is across a left brachiocephalic of the patient. The method can further include generating a notification when the medical device is taking one of the superior vena cava path, the jugular path, or the path that is across a left brachiocephalic of the patient.

In at least one embodiment, a medical device location and tracking system can include a plurality of induction coil assemblies configured to be placed outside of a patient's body. Each induction coil assembly can be configured to generate an electric field and/or a magnetic field (EM field) that permeates at least a portion of the patient's body. At least one EM field can be perpendicular or orthogonal to another EM field. The system can include an inductive sensor configured to be inserted into a patient. The inductive sensor can be configured to generate a sensor signal based on at least one of a magnitude of the EM field, a direction of the EM field, and a flux of the EM field. The system can include an electrical power source configured to energize each induction coil assembly of the plurality of induction coil assemblies in a sequential manner. The system can include a processor configured to receive the sensor signal and determine a location and/or an orientation of the inductive sensor relative to at least one induction coil assembly of the plurality of induction coil assemblies based on an amplitude of the sensor signal and a phase of the sensor signal.

In some embodiments, the plurality of induction coil assemblies includes a first induction coil assembly, a second induction coil assembly, and a third induction coil assembly.

In some embodiments, the inductive sensor is configured to generate a first sensor signal associated with the first induction coil assembly, a second sensor signal associated with the second induction coil assembly, and a third sensor signal associated with the third induction coil assembly.

In some embodiments, the processor is configured to: generate a first amplitude signal and a first phase signal from the first sensor signal, and determine the location and/or an orientation of the inductive sensor relative to the first induction coil assembly based on the first amplitude signal and the first phase sensor signal; generate a second amplitude signal and a second phase signal from the second sensor signal, and determine the location and/or an orientation of the inductive sensor relative to the second induction coil assembly based on the second amplitude signal and the second phase sensor signal; and generate a third amplitude signal and a third phase signal from the third sensor signal, and determine the location and/or an orientation of the inductive sensor relative to the third induction coil assembly based on the third amplitude signal and the third phase sensor signal.

In some embodiments, the processor is configured to: compare magnitudes of the first amplitude signal, the second amplitude signal, and the third amplitude signal to determine the location and/or an orientation of the inductive sensor relative to the first induction coil assembly, the second induction coil assembly, and the third induction coil assembly; and compare phases of the first phase signal, the second phase signal, and the third phase signal to determine the location and/or an orientation of the inductive sensor relative to the first induction coil assembly, the second induction coil assembly, and the third induction coil assembly.

In some embodiments, the processor is configured to generate a coordinate system so that: the relative magnitudes of the first amplitude signal, the second amplitude signal, and the third amplitude signal provide coordinate points on the coordinate system; and the relative phases of the first phase signal, the phase amplitude signal, and the third phase signal provide coordinate points on the coordinate system.

In some embodiments, the electrical power source is configured to energize each induction coil assembly of the plurality of induction coil assemblies via a sinusoidal wave function.

Some embodiments can include a platform configured to be positioned outside a patient's body, wherein the plurality of induction coil assemblies is disposed in and/or on the platform. The platform can have a T-shaped structure. The T-shaped structure can include: an arm having a first arm end and a second arm end; and a stem having a first stem end and a second stem end. The plurality of induction coil assemblies can include a first induction coil assembly disposed in and/or on the arm at or near the first arm end, a second induction coil assembly disposed in and/or on the arm at or near the second arm end, and a third induction coil assembly disposed in and/or on the stem at or near the second stem end.

Further features, aspects, objects, advantages, and possible applications of the present invention will become apparent from a study of the exemplary embodiments and examples described below, in combination with the Figures, and the appended claims.

### BRIEF DESCRIPTION OF THE FIGURES

The above and other objects, aspects, features, advantages and possible applications of the present invention will be more apparent from the following more particular description thereof, presented in conjunction with the following drawings, in which:

FIG. 1 shows an embodiment of the system.

FIG. 2 shows an embodiment of an induction coil assembly that may be used with the system.

FIG. 3 shows another embodiment of an induction coil assembly that may be used with the system.

FIG. 4 shows another embodiment of an induction coil assembly that may be used with the system.

FIG. 5 shows an embodiment of an inductive sensor that may be used with the system.

FIGS. 6A-6B show embodiments of a platform that may be used with the system.

FIG. 7 shows an embodiment the system that includes use of a computer device.

FIG. 8 shows another embodiment of the platform that may be used with the system.

FIGS. 9A-9C show tabular data taken from a simulated test of an embodiment of the system and graphical representations of the data.

FIG. 10 is a display that may be used with an embodiment of the system, showing different possible paths the medical device can take.

FIGS. 11A-11C are displays that may be used with an embodiment of the system, showing different the paths the medical device is taking based on the detected sensor signals.

FIG. 12 shows a module that can be displayed by a computer device with an EM tracking user interface and an ECG tracking user interface.

FIG. 13 shows another embodiment of an induction coil assembly that may be used with the system.

FIGS. 14-15 show other embodiments of the platform that may be used with the system.

FIG. 16 shows a polar coordinate system that can be used to derive location and orientation.

FIGS. 17 and 18 show a superposition of dipoles that can be generated, in an isometric view (FIG. 17) and in a x-y plane projection view (FIG. 18).

### DETAILED DESCRIPTION OF THE INVENTION

The following description is of an embodiment presently contemplated for carrying out the present invention. This description is not to be taken in a limiting sense, but is made merely for the purpose of describing the general principles and features of the present invention. The scope of the present invention should be determined with reference to the claims.

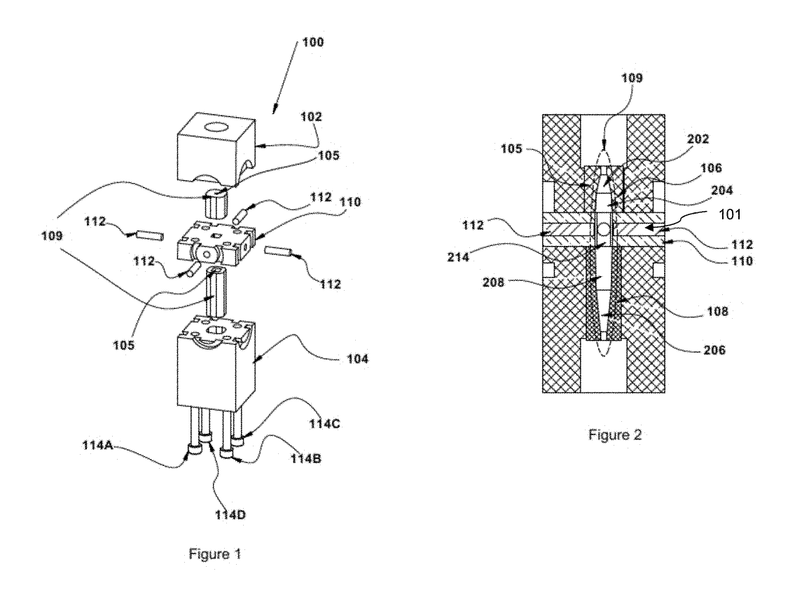

Referring to FIG. 1, embodiments can include a system 100 configured to facilitate determining orientation, proximity, and/or directional movement of a medical device 102. The medical device 102 can be a device that is capable of being inserted into a patient, moved while in the patient, and/or removed from the patient. The system 100 may be used to determine the orientation and/or location of the medical device 102, or at least a portion of it, relative to a portion of the patient's body. The system 100 may be used to determine the direction or path the medical device 102 is taking as the medical device 102 is moved within the patient. Some embodiments can assist in determining the direction or path the medical device 102 will take when the medical device 102 is moved.

Various embodiments disclosed herein may describe use of the system 100 with a medical device 102 that is a catheter 102; however, other medical devices 102 can be used. These can include, but are not limited to cochlear implants, venous access ports, pacemakers, insulin pumps, etc. Embodiments of the catheter 102 may be configured for providing access to a patient's vasculature system (e.g., a venous catheter). The venous catheter 102 may be used to provide treatment for the patient, such as for example hemodialysis or other forms of treatment. Some forms of treatment may include medicine diffusion into the vasculature system via the catheter 102.

The catheter 102 can include a tube 102a inserted into a vein located in the neck (internal jugular vein), chest (subclavian vein or axillary vein), groin (femoral vein), or through veins (basilic vein, cephalic vein and brachial vein) in the arms of a patient. The catheter 102 can include a catheter tip 102b. It is contemplated for the catheter tip 102b to spearhead the insertion of the catheter 102 into the patient. It is also contemplated for the catheter tip 102b to spearhead the movement of the catheter 102 throughout the patient's body as the catheter 102 is maneuvered to a desired location within the patient's vasculature system. The location and/or directional path of the catheter 102 can be useful information for a medical practitioner. As it is contemplated for the catheter tip 102b to spearhead the insertion and subsequent movement of the catheter 102 while in the patient, the location, orientation, and/or directional path of the catheter tip 102b can provide an indication of the location and/or directional path of the catheter 102 itself.

A stylet 103 may be used to guide the catheter 102. For example, a stylet 103 can be attached to a portion of the catheter 102 or inserted through a lumen of the catheter 102. A user can then manipulate the stylet 103 to cause the catheter 102 to move along a vein. The stylet can include a stylet tip 103a. As the stylet 103 can be used to manipulate the catheter 102, the stylet tip 103a can spearhead the insertion of the catheter 102 into the patient. The stylet tip 103a can also spearhead the movement of the catheter 102 throughout the patient's body as the catheter 102 is maneuvered to a desired location within the patient's vasculature system. The location and/or directional path of the stylet tip 103a can be useful information for a medical practitioner. As it is contemplated for the stylet tip 103a to spearhead the insertion and subsequent movement of the catheter 102 while in the patient, the location, orientation, and/or directional path of the stylet tip 103a can provide an indication of the location and/or directional path of the catheter tip 102b and the catheter 102 itself.

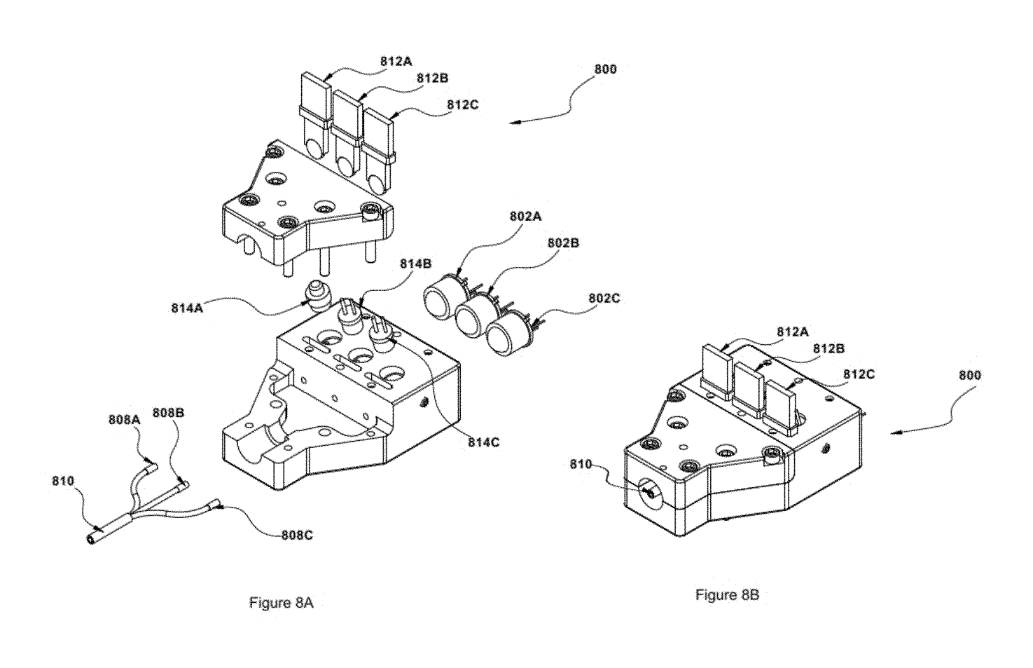

A catheter is typically inserted in a peripheral vein of the patient and advanced proximally toward the heart through increasingly larger veins, until the tip rests in the distal superior vena cava or Cavo atrial junction. As the catheter traverses through the patient's vasculature system, the catheter may take more than one path. For example, at or near a patient's heart and chest, the catheter 102 may follow multiple of paths, which may lead the catheter to or away from the desired location. For example, if inserting the catheter 102 from the right brachial vein, the paths may include a superior vena cava path 1, a jugular path 2, a path that is across the left brachiocephalic 3, etc. (FIG. 10). It is typically desirous for the catheter 102 to take the superior vena cava path 1 for proper and effective treatment (e.g., infusion of medication). With such implementations, the catheter 102 positioned to take the superior vena cava path can be considered to be properly positioned. In addition, the catheter 102 taking the superior vena cava path 1 can be considered to be taking a proper track. The catheter 102 positioned to take, or taking, the jugular path 2, or the path that is across the left brachiocephalic 3 can be considered malpositioned, or taking a maltrack. Thus, it may be beneficial to a medical practitioner to know the position and/or orientation of the catheter 102, as well as the directional path the catheter 102 is taking or will take. Embodiments of the system 100 disclosed herein can not only be used to identify the position and/or orientation of the catheter tip 102b, but it can also identify which direction the catheter tip 102b is taking. Some embodiments can assist in determining the direction the catheter tip 102b will take.

Embodiments of the system 100 can include at least one inductive sensor 104. The inductive sensor 104 can be disposed in and/or on the catheter 102. In at least one embodiment, the inductive sensor 104 can be disposed in and/or on a portion of the catheter 102 at or near the catheter tip 102b. The inductive sensor 104 can generate at least one sensor signal that may be used to determine orientation and/or location of the catheter tip 102b. The sensor signal can also be used to track movement of the catheter tip 102b. It should be noted that the inductive sensor 104 can be placed in and/or on other portions of the catheter 102, such as the tube 102a portion for example. In addition, or in the alternative, the inductive sensor 104 can be placed in and/or on a stylet 103, which may include placing the inductive sensor 104 on the stylet tip 103a.

More than one inductive sensor 104 can be used. For example, an inductive sensor 104 can be used in and/or on at least one of the catheter tip 102b, the catheter tube 102a, the stylet 103, stylet tip 103a, etc. There can be any number of inductive sensors 104 on any portion of the medical device. For example, the catheter tip 102b and/or stylet tip 103a can include one or more inductive sensors 104. The number of inductive sensors 104 in and/or on the catheter tip 102b can be the same as or different from the number of inductive sensors 104 in and/or on the catheter tube 102a or the stylet 103, stylet tip 103a, etc.

The inductive sensor 104 can be an inductive coil, search coil, electronic proximity sensor, magnetometer, etc. configured to generate voltage and/or current (“sensor signal”) based on an electronic field and/or a magnetic field the inductive sensor 104 is placed in and/or in proximity with. The sensor signal generated by the inductive sensor 104 can be a function of the magnitude, change in magnitude, direction, change in direction, flux, and/or change in flux of the electronic field and/or magnetic field.

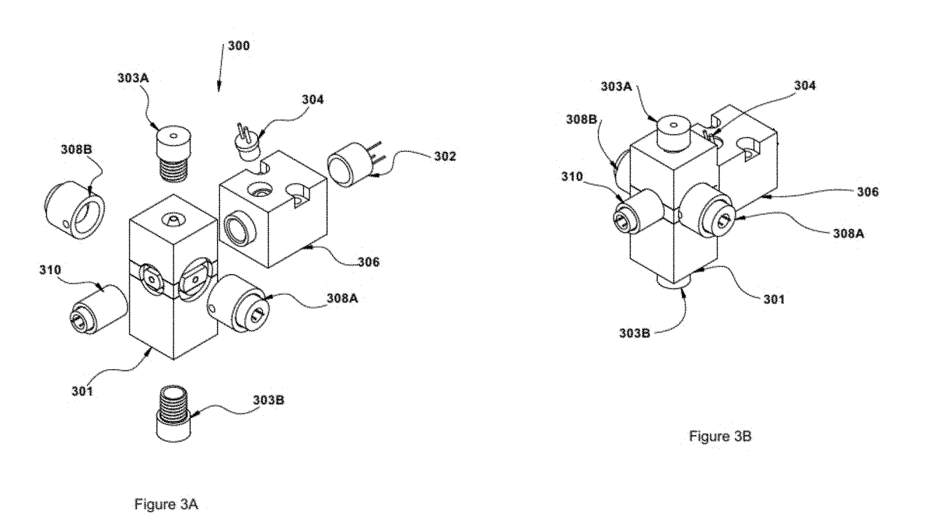

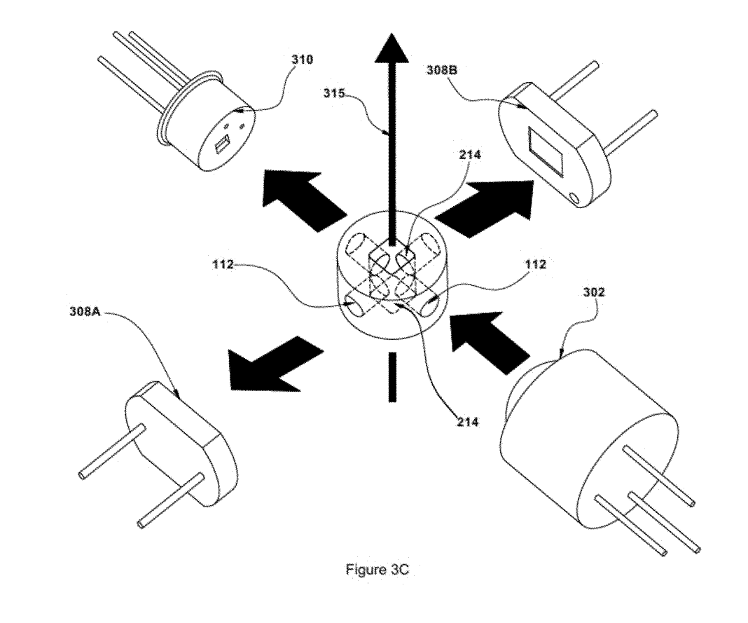

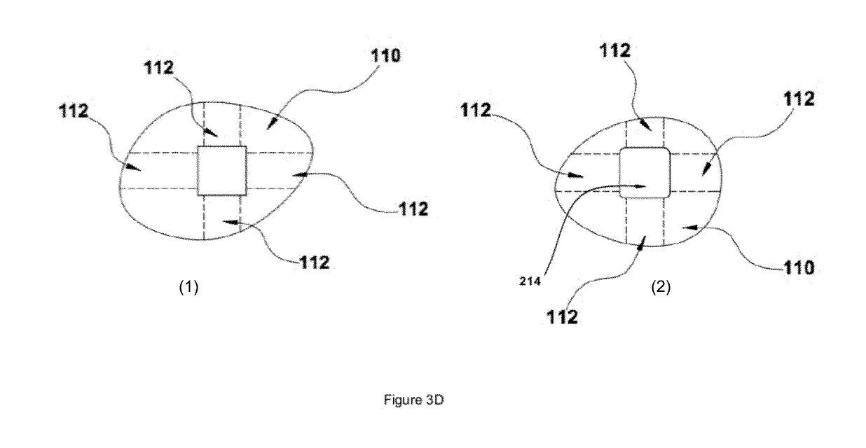

Referring to FIGS. 2-4 and 13, embodiments of the system 100 can include at least one induction coil assembly 106. The induction coil assembly 106 can be used to generate an electronic field and/or magnetic field (“EM field”). The induction coil assembly 106 can be a device that, when energized (e.g., supplied with a voltage or current), generates the EM field having a predetermined magnitude and direction. The EM field can be constant or varying. In some embodiments, the EM field can be used to cause the inductive sensor 104 to generate the sensor signal. For example, the induction coil assembly 106 can be energized to generate the EM field by which the inductive sensor 104 is placed in and/or in proximity with.

Any one or a combination of the induction coil assemblies 106 can include at least one sub-induction coil 108. Some induction coil assemblies 106 can include a plurality of sub-induction coils 108. Any one or a combination of the sub-induction coils 108 in the induction coil assembly 106 can be orientated in a direction that is different from a direction of another sub-induction coil 108. For example, an induction coil assembly 106 may include a first sub-induction coil 108 orientated in an x-direction and a second sub-induction coil 108 in a y-direction. An induction coil assembly 106 may include a first sub-induction coil 108 orientated in a z-direction and a second sub-induction coil 108 in a y-direction. An induction coil assembly 106 may include a first sub-induction coil 108 orientated in a z-direction and a second sub-induction coil 108 in an x-direction. The induction coil assembly 106 can include more than two sub-induction coils 108. For example, an induction coil assembly 106 can include a first sub-induction coil 108 orientated in an x-direction, a second sub-induction coil 108 in a y-direction, and a third sub-induction coil 108 orientated in the z-direction. Any number of sub-induction coils 108 and orientations can be used.

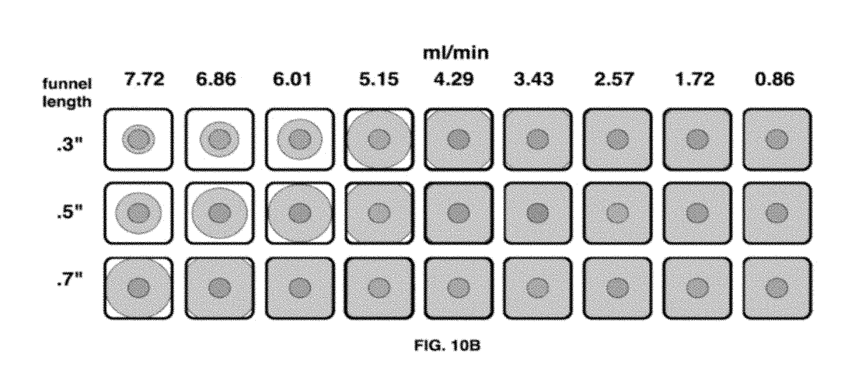

Referring to FIG. 13, in some embodiments, the induction coil assembly 106 can be a coil support structure 107 configured to hold a plurality of induction coils. This can include holding the induction coils at orientations that are different from each other. For example, the coil support structure 107 can be a spherical member having a plurality of coil guides 109. The coil guides 109 can be formed into a surface of the coil support structure 107. Each coil guide 109 can be configured to retain an induction coil. In some embodiments, the coil guide(s) 109 can be configured to retain an induction coil(s) that is/are wound about the coil support structure 107. The positioning and routing of the coil guides 109 can be such that each induction coil retained therein is aligned in a direction that is different from the direction of another induction coil. This can include: a first induction coil being aligned, or generally aligned, in the x-direction; a second induction coil being aligned, or generally aligned, in the y-direction; and a third induction coil being aligned, or generally aligned, in the z-direction. More or less coil guides 109, induction coils, and directional routes can be used.

The sensor signal can be a function of the proximity with which the inductive sensor 104 is relative to the EM field. For example, the inductive sensor 104 can be configured such that the closer the inductive sensor 104 is to the source (e.g., the induction coil assembly 106) of the EM field, the greater in magnitude the sensor signal is generated. The sensor signal can be a function of the direction the inductive sensor 104 is facing relative to the EM field. For example, the inductive sensor 104 can be configured such that when the inductive sensor 104 is parallel to the electric vectors and/or magnetic vectors of the EM field a sensor signal at a first value is generated, and when the inductive sensor 104 is perpendicular or orthogonal to the electric vectors and/or magnetic vectors of the EM field a sensor signal at a second value is generated. The sensor signal first value can be greater or less than the sensor signal second value. The sensor signal can be a function of the amount and/or rate of the electronic field and/or the magnetic field flowing through a given area (e.g., electric flux and/or magnetic flux). For example, the inductive sensor 104 can be configured such that the greater the electric flux and/or magnetic flux detected, the greater the sensor signal that is generated.

In addition, or in the alternative, any one or a combination of the inductive sensors 104 can be configured to generate a sensor signal as a function of a change in any one or a combination of the magnitude of the electric field and/or magnetic field, the direction of the magnitude of the electric field and/or magnetic field, and/or the flux of the magnitude of the electric field and/or magnetic field. For example, the inductive sensor 104 can be configured to generate a signal when the magnitude of the electric field and/or the magnetic field is changing. The inductive sensor 104 can be configured to generate a signal when the direction of the electric field and/or the magnetic field is changing. The inductive sensor 104 can be configured to generate a signal when the flux of the electric field and/or the magnetic field is changing.

The sensor signal(s) can be analyzed to determine the orientation, location, and/or movement of the inductive sensor 104 relative to the induction coil assembly 106. This can be achieved by the inductive sensor 104 being configured to generate a sensor signal as a function of EM magnitude, EM direction, EM flux, and/or changes in any of EM magnitude, EM direction, and/or EM flux. For example, the inductive sensor 104 can be configured to generate a predetermined signal for each of a predetermined EM magnitude, a predetermined EM direction, a predetermined EM flux, a predetermined change in EM magnitude, a predetermined change in EM direction, and/or a predetermined change in EM flux. Each of the predetermined signals can be used to represent the orientation, location, and/or movement of the inductive sensor 104 relative to the induction coil assembly 106.

As a non-limiting example, the inductive sensor 104 may be configured to generate the sensor signal as a function of EM magnitude. For instance, the inductive sensor 104 generating a sensor signal first value at a time i and a sensor signal second value at a time i+1 can be used to determine that the inductive sensor 104 is moving toward or away from the induction coil assembly 106. As an example, if the sensor signal second value is less than the sensor signal first value, this can be an indication that the inductive sensor 104 is moving away from the induction coil assembly 106. Using predetermined currents and voltages to energize the induction coil assembly 106 and a predetermined location of the induction coil assembly 106 can allow for approximating of distance the inductive sensor 104 is from the induction coil assembly 106 based on the magnitude of the EM field generated from that induction coil assembly 106. Thus, the proximity of the inductive sensor 104 to the induction coil assembly 106 can be used to generate a sensor signal that is proportional to the magnitude of the EM field generated, and thus be used to determine the location of the inductive sensor 104. More sensor signal values can be generated at different time points (or distances). Regression of other numerical methods can be used to generate a regression equation to represent the movement. Numerical methods or calculus analyses (e.g., derivative functions) can be used to determine the rate of change the inductive sensor 104 is moving in a certain direction (e.g., toward or away from the induction coil assembly 106). Predictive statistics and other methods can be used to predict movement of the catheter tip 102b and/or stylet tip 103a.

In some embodiments, a plurality of induction coil assemblies 106 can be used. Any one induction coil assembly 106 can be configured to generate an EM field that is the same as or different from an EM field of another induction coil assembly 106. Knowing the EM field and orientation of a first induction coil assembly 106 and those of a second induction coil assembly 106, a third induction coil assembly 106, etc. can allow a user to more accurately determine the position and/or movement of the inductive sensor 104.

As another non-limiting example, the inductive sensor 104 may be configured to generate the sensor signal as a function of EM direction. For instance, the inductive sensor 104 generating a sensor signal first value at a time i and a sensor signal second value at a time i+1 can be used to determine that the inductive sensor 104 is facing parallel (or approximately parallel) or perpendicular or orthogonal (or approximately perpendicular or orthogonal) to the induction coil assembly 106. As an example, if the sensor signal second value is less than the sensor signal first value, this can be an indication that the inductive sensor 104 changed from a parallel (or approximate parallel) orientation to a perpendicular or orthogonal (or approximate perpendicular or orthogonal) orientation relative to the induction coil assembly 106. Using a predetermined orientation of the induction coil assembly 106 and a predetermined location of the induction coil assembly 106 can allow for approximating the angle the inductive sensor 104 is relative to the induction coil assembly 106. Thus, the inductive sensor 104 facing parallel or perpendicular or orthogonal to the EM field of induction coil assembly 106 can be used to generate a sensor signal that is proportional to the direction of the EM field generated, and thus be used to determine the orientation of the inductive sensor 104. More sensor signal values can be generated at different time points (or orientations). Regression of other numerical methods can be used to generate a regression equation to represent the change in orientation. Numerical methods or calculus analyses (e.g., derivative functions) can be used to determine the rate of change the inductive sensor 104 is rotating in a certain direction. Predictive statistics and other methods can be used to predict rotation of the catheter tip 102b and/or stylet tip 103a.

In some embodiments, a plurality of induction coil assemblies 106 can be used. Any one induction coil assembly 106 can be configured to generate an EM field in a direction that is the same as or different from a direction of an EM field of another induction coil assembly 106. Knowing the direction of the EM field of a first induction coil assembly 106 and that of a second induction coil assembly 106, a third induction coil assembly 106, etc. can allow a user to more accurately determine the orientation and/or rotational movement of the inductive sensor 104.

As another non-limiting example, the inductive sensor 104 may be configured to generate the sensor signal as a function of EM flux. For instance, the inductive sensor 104 generating a sensor signal first value at a time i and a sensor signal second value at a time i+1 can be used to determine the angle the inductive sensor 104 is facing relative to the induction coil assembly 106. The inductive sensor 104 may include at least one detection surface 110. The detection surface 110 can be a geometrically planar surface configured to measure the amount of the electric field and/or magnetic field that is normal to the surface area of the detection surface (e.g., measure the electric flux and/or magnetic flux). The inductive sensor 104 can be configured to generate a sensor signal based on the measured flux. For example, the sensor signal may be configured to increase as the flux increases. If the sensor signal second value is different than the sensor signal first value, this can be an indication that the inductive sensor 104 was caused to change its angle relative to the induction coil assembly 106. As an example, if the sensor signal first value is greater than the sensor signal second value, then this may indicate that the inductive sensor 104 rotated from an angle in which the detection surface 110 was more perpendicular or orthogonal to the EM field to an angle in which the detection surface 110 is less perpendicular or orthogonal to the EM field.

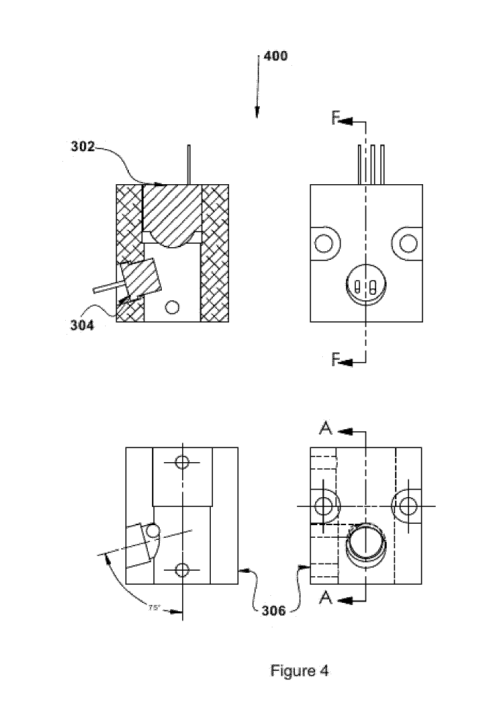

Referring to FIG. 5, the inductive sensor 104 can include a plurality of detection surfaces 110. Any one detection surface 110 can be formed on a geometric plane that is the same as or different from another detection surface 110. Using more than one detection surface 110 can be used to provide a more accurate determination of orientation. Using predetermined voltages and/or currents to energize the induction coil assembly 106, a predetermined orientation of the induction coil assembly 106, a predetermined surface area for the detection surface 110, and a predetermine geometric plane of the detection surface 110 on the induction coil assembly 106 can allow for approximating the orientation of the inductive sensor 104 relative to the induction coil assembly 106. Thus, the inductive sensor 104 having detection surfaces 110 facing normal to or not normal to the EM field of induction coil assembly 106 can be used to generate a sensor signal that is proportional to the direction of the EM field generated, and thus be used to determine the orientation of the inductive sensor 104. More sensor signal values can be generated at different time points (or orientations). Regression of other numerical methods can be used to generate a regression equation to represent the change in orientation. Numerical methods or calculus analyses (e.g., derivative functions) can be used to determine the rate of change the inductive sensor 104 is rotating in a certain direction. Predictive statistics and other methods can be used to predict movement of the catheter tip 102b and/or stylet tip 103a.

In some embodiments, a plurality of induction coil assemblies 106 can be used. Any one induction coil assembly 106 can be configured to generate an EM field in a direction that is the same as or different from a direction of an EM field of another induction coil assembly 106. Knowing the direction of the EM field of a first induction coil assembly 106 and that of a second induction coil assembly 106, a third induction coil assembly 106, etc. can allow a user to more accurately determine the orientation and/or rotational movement of the inductive sensor 104.

Any one or a combination of the proximity, orientation, and/or movement detection and tracking techniques disclosed herein can be used. Any one or a combination of techniques can be used to augment any one or a combination of other techniques or to confirm or validate measurements from other techniques. Any one or a combination of techniques may be more reliable than others in certain situations, and thus those can be selected for use, or the data extracted from those particular techniques used, over other techniques. Any one or a combination of techniques may be more efficient (e.g., require less energy or computational resources) than others, and thus those may be selected for use, or the data extracted from those particular techniques used, over other techniques.

It is contemplated for the operating parameters of the induction coil assembly 106 (e.g., position of, the orientation of, and EM field (magnitude and/or direction) generated by the induction coil assembly 106) to be fixed or held constant during use of the system 100. However, any one or a combination of the operating parameters can be changed or varied during use of the system 100. In some embodiments, any one or a combination of operating parameters can be variable (e.g., dependent on another operating parameter or some other condition).

In at least one embodiment, the induction coil assembly 106 can be positioned outside of a patient. This can include being held in a fixed position outside of a patient. For example, the induction coil assembly 106 can be held at a fixed position on top of or near a patient's chest, neck, leg, etc. In some embodiments, a plurality of induction coil assemblies 106 can be used. Any one induction coil assembly 106 can be held in a position that is in a same geometric plane or a different geometric plane as another induction coil assembly 106. For example, a Cartesian coordinate model can be used with a patient lying in a supine position such that the x-directions are from left to right or right to left across the patient's body (e.g., x-directions can define a coronal plane), the y-directions are from head to toe or toe to head across the patient's body (e.g., y-directions can define a sagittal plane), and the z-directions are from anterior to posterior or posterior to anterior across a patient's body (e.g., z-directions can define a transverse plane). Any one or a combination of induction coil assemblies 106 can be positioned in any one or a combination of the coronal plane, the sagittal plane, and the transverse plane.

A description of a system 100 with two induction coils 106 in various geometric plane positions is provided below. The description of two induction coil assemblies 106 is exemplary, as more or less than two inductions coils 106 can be used. A first induction coil assembly 106 and a second induction coil assembly 106 can be located at the same x-coordinate and y-coordinate but at different z-coordinates. As another example, a first induction coil assembly 106 and a second induction coil assembly 106 can be located at the same x-coordinate and z-coordinate but at different y-coordinates. As another example, a first induction coil assembly 106 and a second induction coil assembly 106 can be located at the same y-coordinate and z-coordinate but at different x-coordinates. Any one induction coil assembly 106 can be orientated along an x-, y-, z-, or intermediate direction that is the same as or different from another induction coil assembly 106. For example, a first induction coil assembly 106 and a second induction coil assembly 106 each can be orientated to be parallel to the x-direction, perpendicular or orthogonal to the y-direction, and perpendicular or orthogonal to the z-direction. As another example, the first induction coil assembly 106 can be orientated to be parallel to the x-direction, perpendicular or orthogonal to the y-direction, and perpendicular or orthogonal to the z-direction, while the second induction coil X can be orientated to be perpendicular or orthogonal to the x-direction, parallel to the y-direction, and perpendicular or orthogonal to the z-direction. Other positions along the x-, y-, and z-directions, other orientations, other numbers of induction coil assemblies 106 can be used.

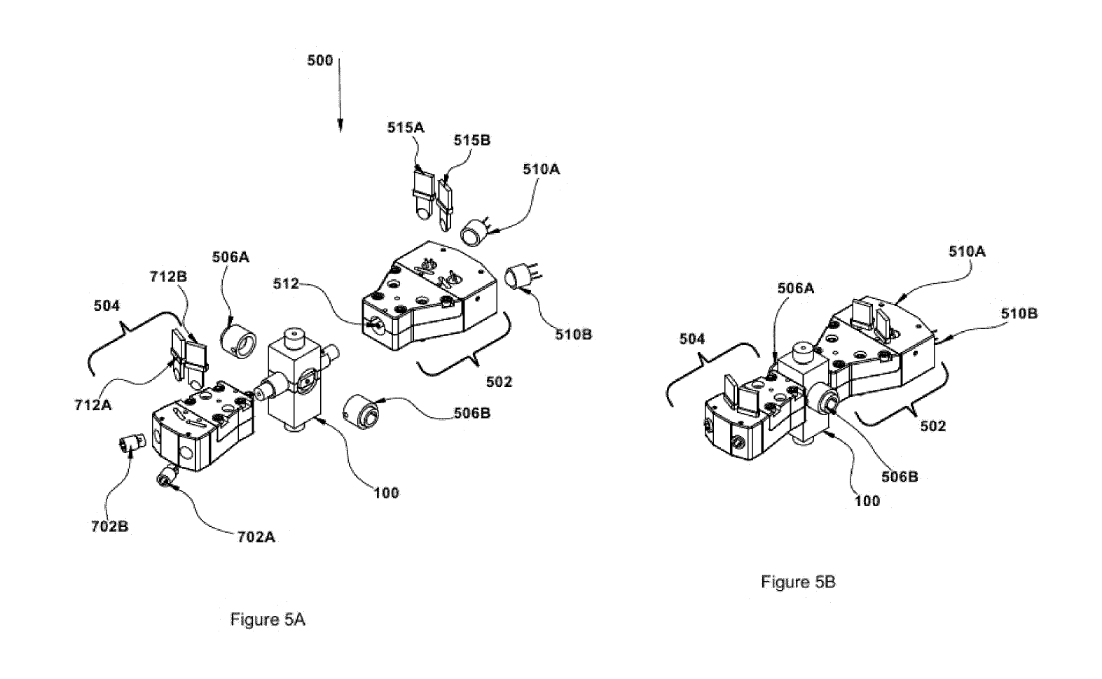

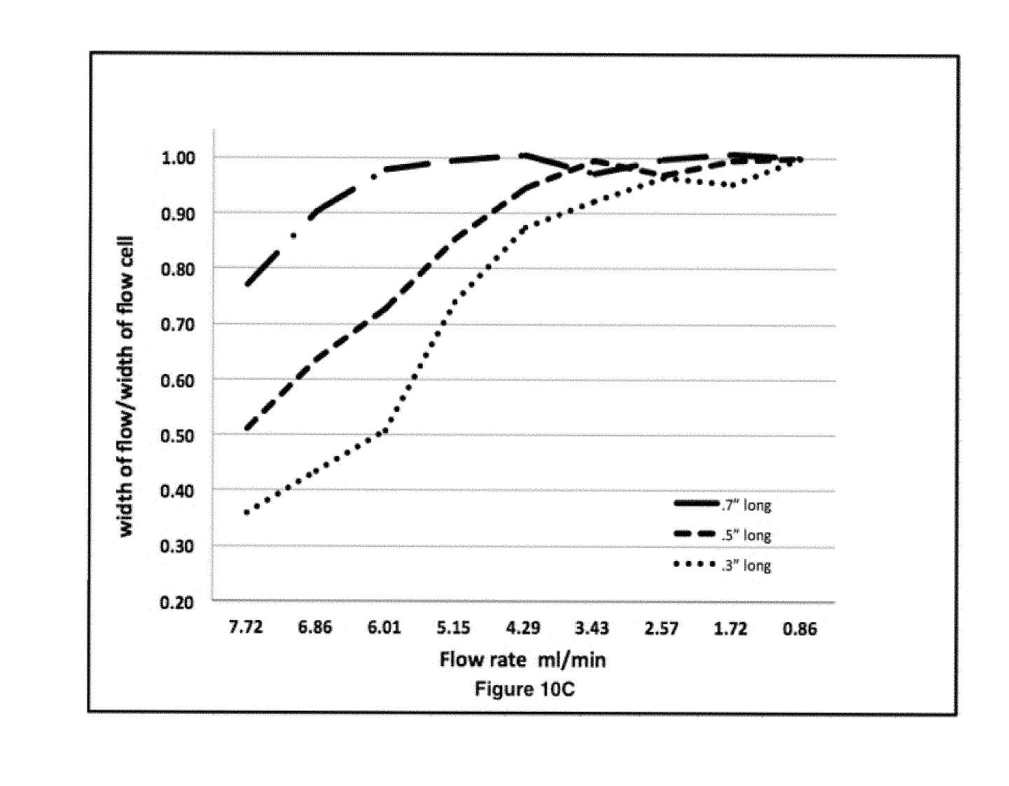

Some embodiments can include at least one platform 112. (See FIGS. 6A, 6B, 14, and 15). The platform 112 can be configured to hold at least one induction coil assembly 106. This can include holding the induction coil assembly 106 outside the patient's body. This can also include holding the induction coil assembly 106 at or near the patient's chest, neck, and/or leg. This can further include holding the induction coil assembly 106 at a predetermined x-, y-, and z-coordinate. This can further include holding the induction coil assembly 106 at a predetermined orientation.

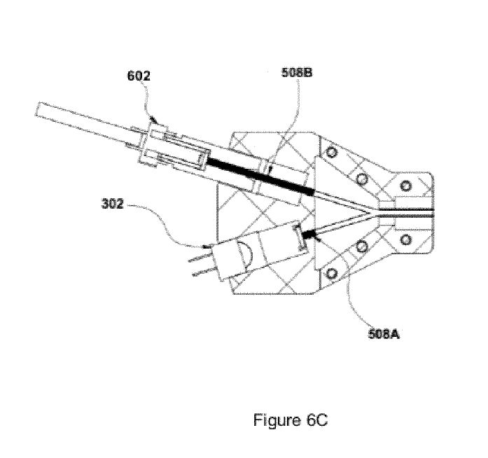

Referring to FIGS. 6-8, embodiments of the platform 112 can include a rigid or semi-rigid structure that can be placed on top of, or held over, a patient. The platform 112 can have a T-shape, but other shapes can be used. This can include a circular shape, diamond shape, a cross-shape, etc. The T-shaped platform 112 can include an arm 114 and a stem 116. The arm 114 can include a first arm end 114a and a second arm end 114b. The stem 116 can include a first stem end 116a and a second stem end 116b. In some embodiments, the T-shaped platform 112 can be placed on top of, or held over, a patient's chest. This may include positioning the platform 112 so that the first arm end 114a is most proximal to the patient's right shoulder, the second arm end 114b is most proximal to the patient's left shoulder, the first stem end 116a is most proximal to the patient's head, and the second stem end 116b is most proximal to the patient's stomach. Other positions and orientations of the platform 112 and the patient (e.g., other than being in a supine position) can be used. It is contemplated for the arm 114 to be within a range from eight inches to fourteen inches in length (from the first arm end 114a to the second arm end 114b). Preferably, the arm 114 is eleven inches in length. It is contemplated for the stem 116 to be within a range from eight inches to fourteen inches in length (from the first stem end 116a to the second stem end 116b). Preferably, the arm 114 is eleven inches in length.

Other shapes of the platform 112 can be used to provide a desired or more comprehensive EM field emission over desired portions of the patient's body. For example, the platform 112 can include additional segments extending from the arm 114 and/or stem 116 to provide induction coil assemblies 106 over certain portions of the patient's neck, leg, or other body part.

The platform 112 can be configured such that the arm 114 and the stem 116 are in a same geometric plane. Alternatively, any portion of the platform 112 can be in a geometric plane that is different from another portion of the platform 112. For example, the stem 116 may have an angled portion that caused the first stem end 116a to be located at a z-coordinate that is different from a z-coordinate of the second stem end 116b.

The platform 112 can include an induction coil assembly 106. In some embodiments, the platform 112 can include a plurality of induction coil assemblies 106. Any one or a combination of induction coil assemblies 106 can be placed along the arm 114 and/or the stem 116. Any one or a combination of induction coil assemblies 106 can be placed in and/or on a portion of the platform 112. Any one or a combination of induction coil assemblies 106 can be placed in the platform 112 while another one or a combination of induction coil assemblies 106 can be placed on the platform 112. A first induction coil assembly 106 can be placed in and/or on the platform 112 so that it is located at a z-coordinate that is the same as or different from a second induction coil assembly 106. For example, the first induction coil assembly 106 can be placed on a bottom surface of the platform 112, and the second induction coil assembly 106 can be placed on a top surface of the platform 112. Similar arrangements can be used for additional induction coil assemblies 106 (e.g., a third induction coil assembly 106, a fourth induction coil assembly 106, etc.).

The inductive sensor 104 can be placed in and/or on a catheter tip 102b and/or stylet tip 103a. The orientation of the inductive sensor 104 with respect to the catheter tip 102b and/or stylet tip 103a can be set so as to provide a reference for a user. For example, the inductive sensor 104 can be configured such that it generates a maximum sensor signal when it is facing parallel to an EM field and a minimum sensor signal when it is facing perpendicular or orthogonal to an EM field. The inductive sensor 104 can also be placed in and/or on the catheter tip 102b and/or stylet tip 103a such that the inductive sensor 104 is parallel to the longitudinal axis 118 of the catheter tip 102b and/or stylet tip 103a. Thus, the inductive sensor 104 can generate a maximum sensor signal when each of the inductive sensor 104 and the catheter tip 102b and/or stylet tip 103a is parallel to the EM field and the inductive sensor 104 can generate a minimum sensor signal when each of the inductive sensor 104 and the catheter tip 102b and/or stylet tip 103a is perpendicular or orthogonal to the EM field. It should be noted that other orientations of the inductive sensor 104 relative to the catheter tip 102b and/or stylet tip 103a can be used, and that making the inductive sensor 104 parallel with the longitudinal axis 118 of the catheter tip 102b and/or stylet tip 103a is for exemplary purposes only.

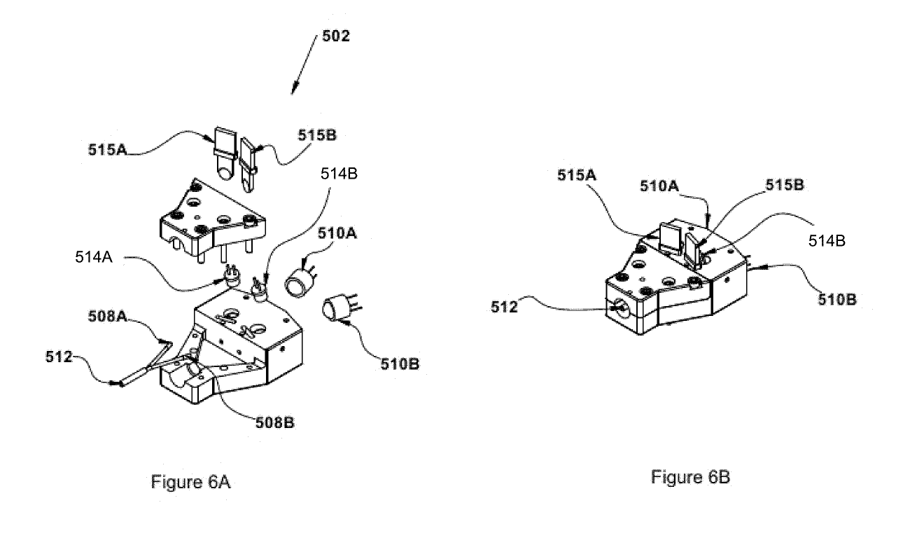

The platform 112 including at least one induction coil assembly 106 can be placed on top of or over the patient. The platform 112 can include electrical connections to supply electrical power to each induction coil assembly 106 from a power source 120 (see FIG. 7). Each induction coil assembly 106 can be energized by causing electrical power to be supplied to them. When energized, each induction coil assembly 106 can generate an EM field. The EM field can permeate through at least a portion of the patient's body. The EM field generated for each induction coil assembly 106 can depend on the orientation, amount of electrical power, and/or the capacity of each induction coil assembly 106. For example, the EM field for an induction coil assembly 106 that is parallel to the z-direction may generate an EM field in the z-direction. The EM field for an induction coil assembly 106 that is perpendicular or orthogonal to the z-direction may generate an EM field in the x- and/or y-directions. The greater the capacity and the greater the amount of electrical power supplied to an induction coil assembly 106 can allow for generating an EM field with greater magnitude.

The magnitude of the EM field generated by an induction coil assembly 106 can be constant due to a constant supply of electrical power. The magnitude of the EM field generated by an induction coil assembly 106 can be varied by varying the electrical power supplied to the induction coil assembly 106. The magnitudes can also be varied by movement of the induction coil assemblies 106 closer and/or further away from the patient. This can be achieved by moving the platform 112 in the z-direction or moving the individual induction coil assemblies 106 within the platform 112. For example, the platform 112 and/or any one or a combination of the induction coil assemblies 106 can be on a track to allow movement.

The direction of the EM field generated by the induction coil assembly 106 can be constant by holding the induction coil assembly 106 in a certain position and orientation. The direction of the EM field generated by the induction coil assembly 106 can also be varied by rotation of the induction coil assemblies 106. This can be achieved by rotating the platform 112 or rotating the individual induction coil assemblies 106 within the platform 112. For example, the platform 112 and/or any one or a combination of the induction coil assemblies 106 can be placed in a gimbal assembly to allow rotational movement.

Referring to FIGS. 1 and 7, some embodiments of the system 100 can include a computer device 122. This can include a processor with an associated non-transitory, non-volatile memory. Examples of a computer device 122 can include a desktop computer, a laptop, a tablet, a smartphone, etc. The computer device 122 can receive, store, and/or process data representative of the sensor signals. For example, the computer device 122 can be in electrical communication (hardwired or wireless via transceivers) with the inductive sensor 104 so as to facilitate transmission of the sensor signals from the inductive sensor 104 to the computer device 122. In some embodiments, the computer device 122 can include analog to digital converters and/or digital to analog converters to transform the sensor signals to and from digital or binary values. In some embodiments, the inductive sensor 104 can include a processor with analog to digital or digital to analog converters to transform the sensor signal to and from digital or binary values before transmitting the sensor signal to the computer device 122.

The computer device 122 can also be in electrical communication (hardwired or wireless via transceivers) with at least one induction coil assembly 106 and the electrical power source. This can allow the computer device to control energizing and/or movement of any one or a combination of induction coil assemblies 106. As noted herein, embodiments of the system 100 can include a plurality of induction coil assemblies 106. The computer device 122 can energize the induction coil assemblies 106 sequentially, simultaneously, or by another scheme. Any one or a combination of the sub-induction coils 108 within any one or a combination of the induction coil assemblies 106 can be energized sequentially, simultaneously, or by another scheme. Energizing sequentially can include supplying electrical power to the induction coil assembly(ies) 106 so as to generate an EM field to a first induction coil assembly 106, then an induction coil assembly 106, then a third induction coil assembly 106 in a logical order. The logical order can follow: energizing the first induction coil assembly 106, then energizing the second induction coil assembly 106, then energizing the third induction coil assembly 106. The logical order can follow: energizing the second induction coil assembly 106, then energizing the first induction coil assembly 106, then energizing the third induction coil assembly 106. The logical order can energize the first induction coil assembly 106 and then de-energize the first induction coil assembly 106 before energizing the second induction coil assembly 106. The logical order can energize the first induction coil assembly 106, then energize the second induction coil assembly 106 before de-energizing the first induction coil assembly 106. Other logical orders can be used.

The computer device 122 may also include operating modules 126 and/or user interfaces 128 that can facilitate control of various components of the system 100, facilitate data manipulation, and/or facilitate data display. This can allow a user to manipulate the sensor signal data and/or display the sensor signal data. The modules can also allow a user to control energizing and/or movement of the induction coil assembly 106.

In at least one embodiment, the system 100 can be used by placing the T-shaped platform 112 including a plurality of induction coil assemblies 106 on a chest of a patient. The catheter 102 having a catheter tip 102b including an inductive sensor 104, or having a stylet 103 with a stylet tip 103a including an inductive sensor 104, can be inserted into a patient. While in the patient, the inductive sensor 104 can be caused to move within or out-from any of the EM fields generated by the induction coil assemblies 106. For example, the inductive sensor 104 can be configured such that when it is in an EM field generated by a first induction coil assembly 106 then the inductive sensor 104 can generate a first sensor signal. The inductive sensor 104 can be configured such that when it is not in the EM field generated by the first induction coil assembly 106 then no first sensor signal is generated. The inductive sensor 104 can be configured such that when the inductive sensor 104 is in an EM field generated by a second induction coil assembly 106 then the inductive sensor 104 can generate a second sensor signal. The inductive sensor 104 can be configured such that when the inductive sensor 104 is not in the EM field generated by the second induction coil assembly 106 then no second sensor signal is generated.

The first induction coil assembly 106 can be configured to generate an EM field at a predetermined magnitude and/or direction. The second induction coil assembly 106 can be configured to generate an EM field at a predetermined magnitude and/or direction that is different from that of the first induction coil assembly 106. This can be done so that the first sensor signal is representative of the inductive sensor 104, and the catheter tip 102b and/or stylet tip 103a, being in the EM field of the first induction coil assembly 106 and the second sensor signal is representative of the inductive sensor 104, and the catheter tip 102b and/or stylet tip 103a, being in the EM field of the second induction coil assembly 106. Similar arrangements can be used for additional induction coil assemblies 106. For example, a third induction coil assembly 106 can be configured to generate an EM field at a predetermined magnitude and/or direction that is different from that of the first and second induction coil assemblies 106, a fourth induction coil assembly 106 can be configured to generate an EM field at a predetermined magnitude and/or direction that is different from that of the first, second, and third induction coil assembly 106, etc.

If a first sensor signal is generated then it can be determined that the inductive sensor 104 is within a predetermined distance (e.g., proximal to) the first induction coil assembly 106. If a second sensor signal is generated then it can be determined that the inductive sensor 104 is within a predetermined distance (e.g., proximal to) the second induction coil assembly 106. If a first and second sensor signal is generated then it can be determined that the inductive sensor 104 is approximately at a location to be within or proximal to the EM fields of both the first induction coil assembly 106 and the second induction coil assembly 106. This may indicate, for example, that the inductive sensor 104 is at a distance that is half-way between the first induction coil assembly 106 and the second induction coil assembly 106. Similar types of analyses can be performed on third sensor signals generated within the EM field of the third induction coil assembly 106, fourth sensor signals generated within the EM field of the fourth induction coil assembly 106, etc.

As the catheter tip 102b and/or stylet tip 103a is moved so does the inductive sensor 104. For example, the inductive sensor 104 may be in the EM field of the first induction coil assembly 106 and then move to the EM field of the second induction coil assembly 106. This can cause the inductive sensor 104 to initially generate a first sensor signal with no second sensor signal, but then generate a second sensor signal with no first sensor signal. This can be an indication that the catheter tip 102b and/or stylet tip 103a has moved from being proximal to the first induction coil assembly 106 to being proximal to the second induction coil assembly 106. Sensor signals can be recorded as a function of time (e.g., sensor signals can be transmitted to the computer device 122 for storage, wherein they can be recorded as a function of time). As noted above, numerical methods and calculus methods can be used to determine the rate at which the inductive sensor 104 is moving. Similar types of analyses can be performed on third sensor signals generated within the EM field of the third induction coil assembly 106, fourth sensor signals generated within the EM field of the fourth induction coil assembly 106, etc.

Sensor signals can also be used to approximate the distance the inductive sensor is to a given induction coil assembly 106. For example, the inductive sensor 104 can generate a first sensor signal due to it being in the EM field of the first induction coil assembly 106. The catheter tip 102b and/or stylet tip 103a can be caused to move towards (generating a sensor signal of greater magnitude) or away (generating a sensor signal of less magnitude) from the first induction coil assembly 106. Multiple first sensor signals can be recorded as a function of time (e.g., sensor signals can be transmitted to the computer device 122 for storage, wherein they can be recorded as a function of time). As noted above, numerical methods and calculus methods can be used to determine the rate at which the inductive sensor 104 is moving. Similar types of analyses can be performed on second sensor signals generated within the EM field of the second induction coil assembly 106, third sensor signals generated within the EM field of the third induction coil assembly 106, fourth sensor signals generated within the EM field of the fourth induction coil assembly 106, etc.

Sensor signals can also be used to approximate the orientation of the inductive sensor relative to a given induction coil assembly 106. For example, the inductive sensor 104 can generate a first sensor signal due to it being in the EM field of the first induction coil assembly 106. The catheter tip 102b and/or stylet tip 103a can be parallel (generating a sensor signal of greater magnitude) or perpendicular or orthogonal (generating a sensor signal of less magnitude). The catheter tip 102b and/or stylet tip 103a can be caused to rotate from being parallel to being perpendicular or orthogonal to the EM field (generating an initial sensor signal of greater magnitude and a subsequent sensor signal of less magnitude) or rotate from being perpendicular or orthogonal to being parallel to the EM field (generating an initial sensor signal of less magnitude and a subsequent sensor signal of greater magnitude). Multiple first sensor signals can be recorded as a function of time (e.g., sensor signals can be transmitted to the computer device 122 for storage, wherein they can be recorded as a function of time). As noted above, numerical methods and calculus methods can be used to determine the rate at which the inductive sensor 104 is rotating. Similar types of analyses can be performed on second sensor signals generated within the EM field of the second induction coil assembly 106, third sensor signals generated within the EM field of the third induction coil assembly 106, fourth sensor signals generated within the EM field of the fourth induction coil assembly 106, etc.

Another means to use sensor signals to approximate the orientation of the inductive sensor relative to a given induction coil assembly 106 can be through measurement of electric field flux and/or magnetic field flux. For example, the inductive sensor 104 can generate a first sensor signal due to it being in the EM field of the first induction coil assembly 106. The catheter tip 102b and/or stylet tip 103a, and a detection surface 110 of the inductive sensor 104, can be normal (generating a sensor signal of greater magnitude) or parallel (generating a sensor signal of less magnitude) to the EM field. The catheter tip 102b and/or stylet tip 103a can be caused to rotate from being parallel to being perpendicular or orthogonal to the EM field (generating an initial sensor signal of less magnitude and a subsequent sensor signal of greater magnitude) or rotate from being perpendicular or orthogonal to being parallel to the EM field (generating an initial sensor signal of greater magnitude and a subsequent sensor signal of less magnitude). Multiple first sensor signals can be recorded as a function of time (e.g., sensor signals can be transmitted to the computer device 122 for storage, wherein they can be recorded as a function of time). As noted above, numerical methods and calculus methods can be used to determine the rate at which the inductive sensor 104 is rotating. Similar types of analyses can be performed on second sensor signals generated within the EM field of the second induction coil assembly 106, third sensor signals generated within the EM field of the third induction coil assembly 106, fourth sensor signals generated within the EM field of the fourth induction coil assembly 106, etc.

Sensor signals can be generated, transmitted, recorded, processed, and/or displayed continuously, periodically, or by some other scheme. The sensor signals can be generated, transmitted, recorded, processed, and/or displayed whether the catheter tip 102b and/or stylet tip 103a is stationary, moving, and/or being rotated. The data generated from the sensor signals can be processed and displayed by the computer device 122. The display of the data can provide the location and/or orientation of the catheter tip 102b and/or stylet tip 103a relative to any one or a combination of induction coil assemblies 106. The display of the data can provide the movement of the catheter tip 102b and/or stylet tip 103a relative to any one or a combination of induction coil assemblies 106. A model of the patient's body with a Cartesian coordinate system can be used to illustrate the location, movement, and/or orientation of the catheter tip 102b and/or stylet tip 103a.

In at least one embodiment, The T-shaped platform 112 can include structure having an arm 114 and a stem 116. A first induction coil assembly 106′ can be located at or near the first arm end 114a. The first induction coil assembly 106′ can be configured to generate an EM field in the z-direction. A second induction coil assembly 106″ can be located at or near the second arm end 114b. The second induction coil assembly 106″ can be configured to generate an EM field in the z-direction. A third induction coil assembly 106″′ can be located at or near the second stem end 116b. The third induction coil assembly 106″′ can be configured to generate an EM field in the z-direction.

Generally, the catheter tip 102b and/or stylet tip 103a, and thus the inductive sensor 104 (if parallel to the longitudinal axis 118), when inserted into the patient and maneuvered in the patient are in the x- and y-directions. The general perpendicular or orthogonal orientation between the inductive sensor 104 and the EM fields generated by the first, second, and third induction coil assemblies 106 can cause the inductive sensor 104 to generate weaker signals than if the relative orientations between them were parallel, but the perpendicular or orthogonal orientation can allow for obtaining a sensor signal as a function of relative distance. For example, if the first, second, and third induction coil assemblies 106′, 106″, 106″′ were energized sequentially, then a separate sensor signal can be generated for each of the first, second, and third induction coil assembly 106′, 106″, 106″′. The sensor signal strength for each sensor signal would be a function of the distance the inductive sensor 104 is from the respective induction coil assembly 106′, 106″, 106″′.

In addition to the first, second, and third induction coil assemblies 106′, 106″, 106″′ located at or near the first arm end 114a, the second arm end 114b, and the second stem end 116b, inter-disposed induction coil assemblies 106″″ can be located on the T-shaped platform 112. For example, at least one inter-disposed induction coil assembly 106″″ can be located in and/or on the T-shaped platform 112 at a point along the arm 114 that is between the first arm end 114a and second arm end 114b. In addition, or in the alternative, at least one inter-disposed induction coil assembly 106″″ can be located in and/or on the T-shaped platform 112 at a point along the stem 116 that is between the first stem end 116a and second stem end 116b. Any one or a combination of the inter-disposed induction coil assemblies 106″″ can be configured to generate an EM field in the x- or y-directions. In at least one embodiment, the inter-disposed induction coil assemblies 106″″ located in the arm 114 can be configured to generate EM fields in the x-direction. In at least one embodiment, the inter-disposed induction coil assemblies 106″″ located in the stem 116 can be configured to generate EM fields in the y-direction.

As noted above, the catheter tip 102b and/or stylet tip 103a, and thus the inductive sensor 104, when inserted into the patient and maneuvered in the patient is in the x- and y-directions. The general parallel orientation between the inductive sensor 104 and the EM fields generated by the inter-disposed induction coil assemblies 106 can cause the inductive sensor to generate stronger signals than if the relative orientations between them were perpendicular or orthogonal. The stronger signals can be used to better identify movement of the catheter tip 102b and/or stylet tip 103a in the x- and y-directions.

The first, second, and third induction coil assemblies 106′, 106″, 106″′ can be configured to generate EM fields in a direction that is different from the EM fields generated by the inter-disposed induction coil assemblies 106″″. This can allow the inter-disposed induction coil assemblies 106″″ to cause the inductive sensor 104 to generate more intense sensor signals due to the parallel orientation of the EM field generated by them relative to the inductive sensor 104. Using ratios for the sensor signals to determine the catheter path, the system 100 can operate in an efficient and computationally minimal manner.

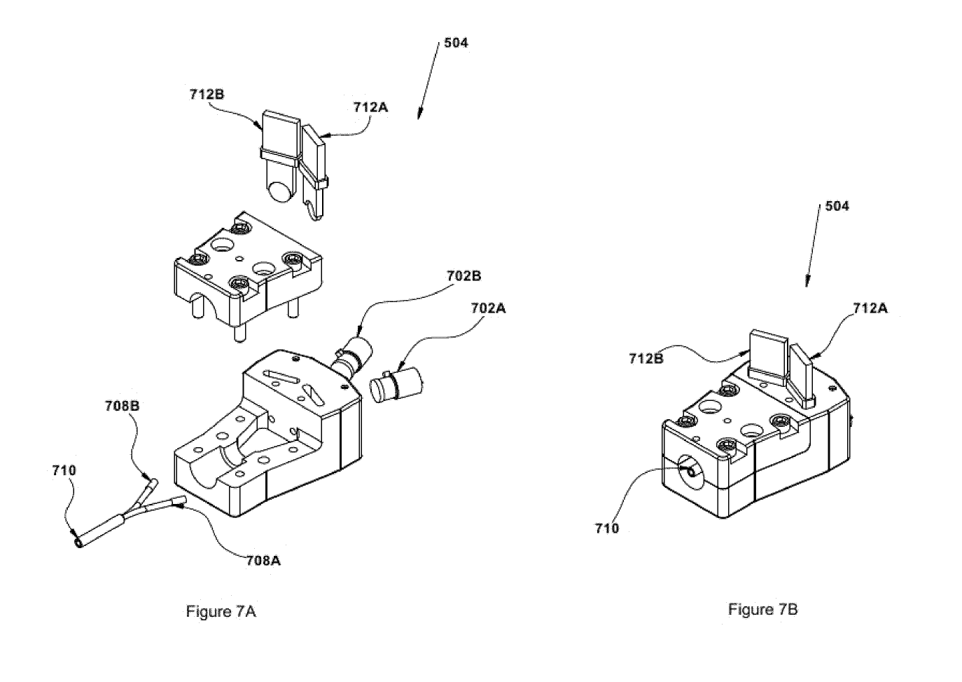

An exemplary embodiment of the system 100 was used to generate location and tracking data to demonstrate the effectiveness of the system 100. In this exemplary embodiment, the T-shaped platform 112 was provided with a first induction coil assembly 106′ at the first arm end 114a, a second induction coil assembly 106″ at the second arm end 114b, and a third induction coil assembly 106″′ at the second stem end 116b (denoted as “Left”, “Right”, and “Bottom”, respectively, in FIGS. 9A-C). Each of the first, second, and third induction coil assemblies 106′, 106″, 106″′ were arranged to generate an EM field in a z-direction. Ten inter-disposed induction coil assemblies 106″″ were positioned along the arm 114 between the first arm end 114a and the second arm end 114b, each arranged to generate an EM field in a x-direction (denoted as 0, 1, 2 . . . 9, respectively, in FIGS. 9A-C). Eight inter-disposed induction coil assemblies 106″″ were positioned along the stem 116 between the first stem end 116a and the second stem end 116b, each arranged to generate an EM field in a y-direction (denoted as 0, −1, −2 . . . −7, respectively, in FIGS. 9A-C). In the exemplary embodiment, the platform was positioned to be approximately three inches above the inductive sensor 104. A body dummy with a simulated vasculature system was used and the inductive sensor 104 was attached to a stylet. The stylet was moved throughout the simulated vasculature system. The stylet was moved through a simulated superior vena cava path 1, a simulated jugular path 2, and a simulated subclavian path. Sensor signals for each path were recorded.

As can be seen from FIGS. 9A-9C, sensor signals were collected from the inductive sensor 104 being in or in proximity to the EM fields generated by each coil assembly 106. The signals from the inter-disposed induction coil assemblies 106″′ were plotted on the line graphs shown in FIGS. 9A-9C. The signals from the first, second, and third induction coil assemblies 106′, 106″, 106″ were plotted on the triangular positioning charts shown in FIGS. 9A-9C. The line graphs and triangular positioning chart of FIG. 9A demonstrate that the inductive sensor 104 was most proximal to the second stem end 114b and slightly towards the first arm end 116a. The line graphs and triangular positioning chart of FIGS. 9B and 9C demonstrate that the inductive sensor 104 was most proximal to a position that was half-way between the first stem end 116a and the second stem end 116b and half-way between the first arm end 114a and the second arm end 114b.

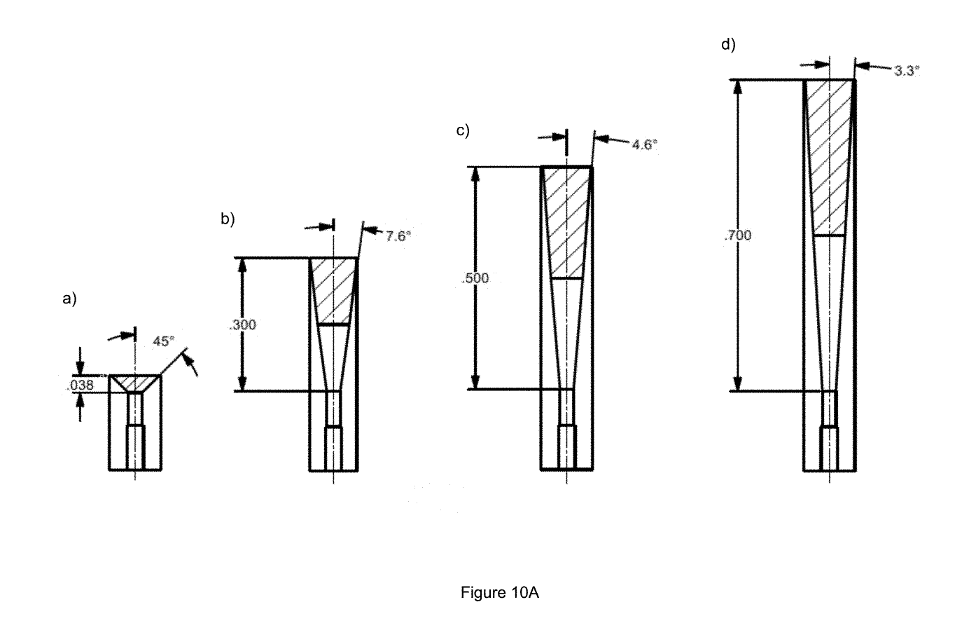

In at least one embodiment, electrocardiograph (“ECG”) signals can be used to further assist with location, orientation, and/or tracking of the catheter tip 102b and/or stylet tip 103a. (See FIG. 12). For example, ECG signals can be transmitted to the computer device 122 and displayed via a user interface 128. Inflection points of waveforms (e.g., P-waves) can be used to determine the relative distance between the catheter tip 102b and/or stylet tip 103a and the heart. For example, a pair of ECG electrodes can be placed on the patient. Another ECG sensor electrode can be placed in and/or on the catheter tip 102b and/or stylet tip 103a. The electrodes can be energized to produce ECG signals. These signals can be recorded and transmitted to an electrocardiogram display. As the catheter tip 102b and/or stylet tip 103a is moved away from or towards the patient's heart, changes in the P-wave of the ECG signals can be observed. This can provide an indication that the catheter tip 102 b and/or stylet 103a is moving towards or away from the heart.

In at least one embodiment, an ultrasound probe can be used to further assist with location, orientation, and/or tracking of the catheter tip 102b and/or stylet tip 103a. The ultrasound probe can be configured to emit ultrasound pulses and receive echo pulses. A transducer array may be included in the ultrasound probe to receive the echo pulses and to facilitate generation of ultrasound data (e.g., an ultrasound image) related to the position of the catheter tip 102b and/or stylet tip 103a. The ultrasound signals can be transmitted to the computer device 122 and displayed via a user interface.

Referring to FIGS. 10-12, as noted herein, the system can include use of at least one computer device 122. Program logic and/or application software can be used to generate at least one module 126 via the computer device 122. The computer device 122 can further include a display monitor configured to display the module 126 or aspects of the module (e.g., user interfaces 128, actual indicia 130, etc.). In some embodiments, the module 126 can be programmed to generate a user interface 128, a display screen 124, etc. At least one embodiment of the display screen 124 can include a silhouette of a patient. The silhouette can include anticipated paths for the medical device to take. For example, the display screen can show a display screen 124 including the superior vena cava path 1, the jugular path 2, and/or the path that is across a left brachiocephalic 3 of the patient. Some embodiments may only display the correct path or the proper track (e.g., the superior vena cava path 1) that is expected for the medical device to take. Some embodiments may display all of the paths and highlight (e.g., cause it to blink, cause it to be a different color, etc.) the correct path or proper track that is expected for the medical device to take.

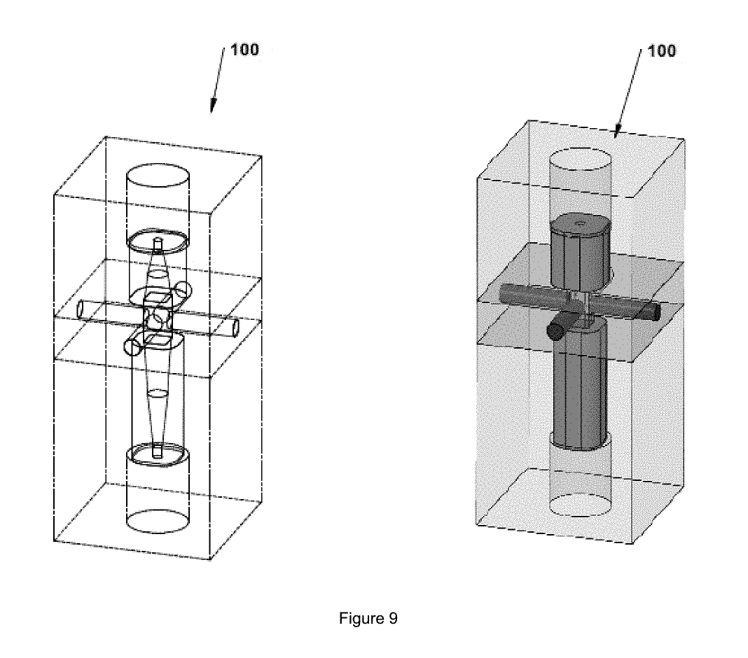

In some embodiments, no path will be displayed until the catheter tip 102b and/or stylet tip 103a is detected and/or its projected track or movement is determined. For example, if the location of the catheter tip 102b and/or stylet tip 103a is determined to be properly positioned and/or taking a proper track (e.g., taking the superior vena cava path 1), then the superior vena cava path 1 will be displayed to indicate that the proper path is being taken, as shown in FIG. 11A. If the location of the catheter tip 102b and/or stylet tip 103a is determined to be malpositioned and/or taking a maltrack (e.g., the jugular path 2 and/or the path that is across a left brachiocephalic 3 of the patient) then the jugular path 2 and/or the path that is across a left brachiocephalic 3 of the patient will be displayed to indicate that the improper path is being taken, as shown in FIGS. 11B-11C.

Referring to FIG. 12, in some embodiments, the computer device 122 can be programmed to generate various tracking modules. These can include tracking modules associated with the various means to track the position and movement of the medical device. For example, the modules can include an EM tracking module 126, an ECG tracking module 126, an ultrasound tracking module 126, etc. Each module 126 can be programmed to generate a user interface 128 to facilitate command and control of at least one component of the system 100. For example, the EM tracking user interface 128 can be programmed to generate actionable indicia 130 to allow a user to manipulate the sensor signal data and/or display the sensor signal data. The actionable indicia 130 can also allow a user to control energizing and/or movement of the induction coil assembly(ies) 106. As another example, the ECG tracking user interface 128 can be programmed to generate actionable indicia 130 to allow a user to manipulate the ECG data and/or display the ECG signal data. The actionable indicia 130 can also allow a user to control energizing the ECG electrodes. As another example, the ultrasound tracking user interface 128 can be programmed to generate actionable indicia 130 to allow a user to manipulate the ultrasound data and/or display the ultrasound signal data.

In at least one embodiment, the computer device 122 can include a module 126 that displays multiple user interfaces 128. For example, the module 126 can be programmed to display any one or a combination of the EM tracking user interface 128, the ECG tracking user interface 128, and the ultrasound user interface 128. As an exemplary embodiment, FIG. 12 shows a module 126 that includes an EM tracking user interface 128 and an ECG tracking user interface 128. In this exemplary embodiment, the EM tracking portion of the system 100 can be used to determine that the catheter tip 102b and/or stylet tip 103a is properly positioned and is taking a proper track by showing the superior vena cava path 1. Once the medical practitioner is comfortable that the proper track is being taken, the ECG tracking portion and/or the ultrasound tracking portion of the system 100 can be used to augment the positioning and/or tracking. In addition, or in the alternative, the ECG tracking portion and/or the ultrasound tracking portion of the system 100 can be used to provide more accurate estimates of the position, orientation, and/or tracking of the catheter tip 102b and/or stylet tip 103a.

An embodiment of the system 100 can be configured to determine a location and/or an orientation of the inductive sensor 104 relative to an induction coil assembly 106 based on an amplitude of a sensor signal and a phase of the sensor signal. For example, a plurality of induction coil assemblies 106 can be placed outside of a patient's body. Each induction coil assembly 106 can be configured to generate an electric field and/or a magnetic field (EM field) that permeates at least a portion of the patient's body. The system 100 can be configured so that at least one EM field is perpendicular or orthogonal to another EM field. An inductive sensor 104 can be inserted into a patient. The inductive sensor 104 can be configured to generate a sensor signal based on at least one of a magnitude of the EM field, a direction of the EM field, and a flux of the EM field. An electrical power source 120 can be used to energize each induction coil assembly 106 in a sequential manner. This can be via a sinusoidal wave function (e.g., a 100 KHz sine wave function). A processor (e.g., the computer device 122) can be used to receive the sensor signal and determine a location and/or an orientation of the inductive sensor 104 relative to at least one induction coil assembly 106 based on an amplitude of the sensor signal and a phase of the sensor signal.

In an exemplary embodiment, the plurality of induction coil assemblies 106 can include a first induction coil assembly 106, a second induction coil assembly 106, and a third induction coil assembly 106. The inductive sensor 104 can be configured to generate a first sensor signal associated with the first induction coil assembly 106, a second sensor signal associated with the second induction coil assembly 106, and a third sensor signal associated with the third induction coil assembly 106.

The computer device 122 can be configured to generate a first amplitude signal and a first phase signal from the first sensor signal and determine the location and/or an orientation of the inductive sensor relative to the first induction coil assembly based on the first amplitude signal and the first phase sensor signal. The computer device 122 can generate a second amplitude signal and a second phase signal from the second sensor signal and determine the location and/or an orientation of the inductive sensor 104 relative to the second induction coil assembly 106 based on the second amplitude signal and the second phase sensor signal. The computer device 122 can generate a third amplitude signal and a third phase signal from the third sensor signal and determine the location and/or an orientation of the inductive sensor 104 relative to the third induction coil assembly 106 based on the third amplitude signal and the third phase sensor signal.

This can be achieved by the computer device 122 comparing magnitudes of the first amplitude signal, the second amplitude signal, and the third amplitude signal to determine the location and/or an orientation of the inductive sensor 104 relative to the first induction coil assembly 106, the second induction coil assembly 106, and the third induction coil assembly 106. In addition, or in the alternative, the computer device 122 can compare phases of the first phase signal, the second phase signal, and the third phase signal to determine the location and/or an orientation of the inductive sensor 104 relative to the first induction coil assembly 106, the second induction coil assembly 106, and the third induction coil assembly 106.

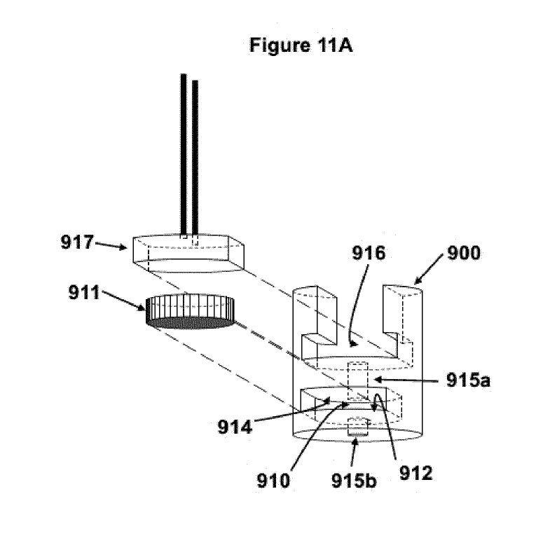

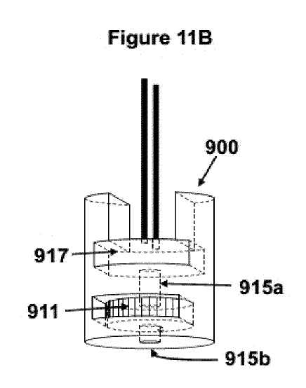

As noted herein, a Cartesian coordinate system can be used to identify locations and orientations of the inductive sensor 104 relative to any of the induction coil assemblies 106. Thus, the computer device 122 can be configured to generate a coordinate system so that the relative magnitudes of the first amplitude signal, the second amplitude signal, and the third amplitude signal provide coordinate points on the coordinate system. In addition, or in the alternative, the relative phases of the first phase signal, the phase amplitude signal, and the third phase signal can be used to provide coordinate points on the coordinate system. As an example, FIG. 16 shows a polar coordinate system that can be used to derive location and orientation. FIGS. 17 and 18 show a superposition of dipoles that can be generated, in an isometric view (FIG. 17) and in a x-y plane projection view (FIG. 18).

The system 100 can include use of any of the platform 112. Again, the platform 112 can be a T-shaped structure 112. A first induction coil assembly 106 can be disposed in and/or on the arm 114 at or near the first arm end 114a. A second induction coil assembly 106 can be disposed in and/or on the arm 114 at or near the second arm end 114b. A third induction coil assembly 106 can be disposed in and/or on the stem 116 at or near the second stem end 116b.

It should be understood that modifications to the embodiments disclosed herein can be made to meet a particular set of design criteria. For instance, any of the induction coil assemblies 106, sub-induction coils 108, inductive sensors 104, platforms 112, computer devices 122, or any other component of the system 100 can be any suitable number or type of each to meet a particular objective. Therefore, while certain exemplary embodiments of the system 100 and methods of using the same disclosed herein have been discussed and illustrated, it is to be distinctly understood that the invention is not limited thereto but may be otherwise variously embodied and practiced within the scope of the following claims.

It should also be appreciated that some components, features, and/or configurations may be described in connection with only one particular embodiment, but these same components, features, and/or configurations can be applied or used with many other embodiments and should be considered applicable to the other embodiments, unless stated otherwise or unless such a component, feature, and/or configuration is technically impossible to use with the other embodiment. Thus, the components, features, and/or configurations of the various embodiments can be combined together in any manner and such combinations are expressly contemplated and disclosed by this statement.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

What is claimed is:

1. A medical device location and tracking system, comprising:
a plurality of induction coil assemblies configured to be placed outside of a patient's body, wherein:
each induction coil assembly is configured to generate an electric field and/or a magnetic field (EM field) that permeates at least a portion of the patient's body when energized; and
each induction coil assembly, comprising at least two orthogonal sub-coils;
an inductive sensor configured to be inserted into a patient, wherein the inductive sensor is configured to generate a sensor signal in response to the energization of each of the plurality of induction coil assemblies;
an electrical power source configured to energize each induction coil assembly of the plurality of induction coil assemblies in a sequential manner; and
a processor configured to receive the sensor signal and determine a location and/or an orientation of the inductive sensor relative to at least one induction coil assembly of the plurality of induction coil assemblies based on an amplitude of the sensor signal and a phase of the sensor signal.

2. The system recited in claim 1, wherein the plurality of induction coil assemblies comprises a first induction coil assembly, a second induction coil assembly, and a third induction coil assembly.

3. The system recited in claim 2, wherein the inductive sensor is configured to generate a first sensor signal associated with the first induction coil assembly, a second sensor signal associated with the second induction coil assembly, and a third sensor signal associated with the third induction coil assembly.

4. The system recited in claim 3, wherein the processor is configured to:
generate a first amplitude signal and a first phase signal from the first sensor signal, and determine the location and/or an orientation of the inductive sensor relative to the first induction coil assembly based on the first amplitude signal and the first phase sensor signal;
generate a second amplitude signal and a second phase signal from the second sensor signal, and determine the location and/or an orientation of the inductive sensor relative to the second induction coil assembly based on the second amplitude signal and the second phase sensor signal; and
generate a third amplitude signal and a third phase signal from the third sensor signal and determine the location and/or an orientation of the inductive sensor relative to the third induction coil assembly based on the third amplitude signal and the third phase sensor signal.

5. The system recited in claim 4, wherein the processor is further configured to:
compare magnitudes of the first amplitude signal, the second amplitude signal, and the third amplitude signal to determine the location and/or an orientation of the inductive sensor relative to the first induction coil assembly, the second induction coil assembly, and the third induction coil assembly; and
compare phases of the first phase signal, the second phase signal, and the third phase signal to determine the location and/or an orientation of the inductive sensor relative to the first induction coil assembly, the second induction coil assembly, and the third induction coil assembly.

6. The system recited in claim 5, wherein the processor is configured to generate a coordinate system so that:
the relative magnitudes of the first amplitude signal, the second amplitude signal, and the third amplitude signal provide coordinate points on the coordinate system; and
the relative phases of the first phase signal, the phase amplitude signal, and the third phase signal provide coordinate points on the coordinate system.

7. The system recited in claim 2, wherein each of the plurality of induction coil assemblies comprising three orthogonal sub-coils.

8. The system recited in claim 3, wherein each of the plurality of induction coil assemblies and each of the orthogonal sub-coils are energized sequentially.

9. The system recited in claim 7, wherein the processor is configured to generate an amplitude signal and a phase signal from the induction sensor response to the sequential energizing of each of the orthogonal sub-coil of each of the plurality of induction coil assemblies.

10. The system recited in claim 9, wherein the processor is further configured to:
compare each amplitude signals and each of the phase signals from the induction sensor response to the sequential energizing of each of the orthogonal sub-coil of each of the plurality of induction coil assemblies.

11. The system recited in claim 10, wherein the processor is configured to generate a coordinate system and relative location of the medical device based on each amplitude signals and each of the phase signals from the induction sensor response to the sequential energizing of each of the orthogonal sub-coil of each of the plurality of induction coil assemblies.

12. The system recited in claim 1, wherein the electrical power source is configured to energize each induction coil assembly of the plurality of induction coil assemblies via a sinusoidal wave function.

13. The system recited in claim 1, further comprising a platform configured to be positioned outside a patient's body, wherein the plurality of induction coil assemblies is disposed in and/or on the platform.

14. The system recited in claim 13, wherein the platform comprises a T-shaped structure, wherein the T-shaped structure comprises:
an arm having a first arm end and a second arm end; and
a stem having a first stem end and a second stem end;
wherein the plurality of induction coil assemblies comprises a first induction coil assembly disposed in and/or on the arm at or near the first arm end, a second induction coil assembly disposed in and/or on the arm at or near the second arm end, and a third induction coil assembly disposed in and/or on the stem at or near the second stem end.

15. The system recited in claim 1, further comprising:
a pair of electrocardiograph (“ECG”) electrodes placed on the patient's body;
an ECG sensor electrode placed in and/or on the medical device;
wherein the pair of ECG electrodes and the ECG sensor electrode are configured to produce ECG signals that are transmitted to an electrocardiogram display,
wherein changes in a P-wave of the ECG signals indicate movement of the medical device towards or away from a heart of a patient.

16. The system recited in claim 15, wherein the inductive sensor is deposited at one end of a metal wire, wherein the metal wire also serves as the ECG sensor electrode.

17. A medical device location and tracking system, comprising:
a platform configured to be positioned outside a patient's body, the platform comprising:
a first induction coil assembly, a second induction coil assembly, a third induction coil assembly;
wherein each of the first induction coil assembly, the second induction coil assembly, the third induction coil assembly, and the inter-disposed induction coil assembly is configured to generate an electric field and/or a magnetic field (EM field) that permeates at least a portion of the patient's body;
wherein at least one EM field of the inter-disposed induction coil assembly is perpendicular or orthogonal to at least one EM field of the first induction coil assembly, the second induction coil assembly, the third induction coil assembly;
a power source configured to energize at least two of the first induction coil assembly, the second induction coil assembly, the third induction coil assembly, and the inter-disposed induction coil assembly in a sequential manner; and
an inductive sensor disposed in and/or on a portion of a medical device configured to be inserted into the patient, wherein the inductive sensor is configured to generate a sensor signal based on being within and/or proximal to any one or a combination of the EM fields generated by the first induction coil assembly, the second induction coil assembly, the third induction coil assembly, and the inter-disposed induction coil assembly.

18. A method for locating and tracking a medical device, comprising:
positioning a plurality of induction coil assemblies on top of and/or over a patient's body;
generating a plurality of electric fields and/or magnetic fields (EM fields) that permeate at least a portion of the patient's body by energizing at least one of the induction coil assemblies;
inserting the medical device with an inductive sensor into the patient;
determining a location of the medical device based on a proximity with which the inductive sensor is relative to at least one of induction coil assemblies;
tracking movement of the medical device to determine if the medical device is taking a superior vena cava path, a jugular path, or a path that is across a left brachiocephalic of the patient.

19. The method of claim 18, further comprising:
generating a notification when the medical device is taking one of the superior vena cava path, the jugular path, or the path that is across a left brachiocephalic of the patient.

20. The method of claim 19, wherein the notification when the medical device is taking one of the superior vena cava path is different from the notification when the medical device is taking the jugular path, or the path that is across a left brachiocephalic of the patient.
